(19) United States
(12) Patent Application Publication
(10) Pub. No.: US 2008/0172091 A1
(43) Pub. Date: Jul. 17, 2008

(54) Spinal Stabilization System

(75) Inventor: Kent M. Anderson, Memphis, TN (US)

Correspondence Address: HAYNES AND BOONE, LLP, 901 Main Street, Suite 3100, Dallas, TX, 75202, US

(73) Assignee: WARSAW ORTHOPEDIC, INC., Warsaw, IN (US)

(21) Appl. No.: 11/622,967

(22) Filed: Jan. 12, 2007

(51) Int. Cl. A61B 17/58

(52) U.S. Cl. 606/246; 606/254

(57) ABSTRACT

A system for stabilizing the spine, according to which a first dampening member is compressed in response to compressive loads on the spine, and a second dampening member is compressed in response to tensile loads on the spine.

### BACKGROUND

The present invention relates to a system for stabilizing the human spine.

Intervertebral discs that extend between adjacent vertebrae in vertebral columns of the human body provide critical support between the adjacent vertebrae while permitting multiple degrees of motion. These discs can rupture, degenerate, and/or protrude by injury, degradation, disease, or the like, to such a degree that the intervertebral space between adjacent vertebrae collapses as the disc loses at least a part of its support function, which can cause impingement of the nerve roots and severe pain.

Some of the current procedures for treating this malady involve pedicular systems for dynamic stabilization of the vertebrae that include a viscoelastic dampening member to allow motion in compression. However, these systems are not flexible, or compliant, in tension, and therefore produce asymmetric flexion-extension biomechanics which is undesirable.

The present invention is directed to an improved system of the above type that allows motion in compression and tension and produces symmetric flexion-extension biomechanics. Various embodiments of the invention may possess one or more of the above features and advantages, or provide one or more solutions to the above problems existing in the prior art.

### BRIEF DESCRIPTION OF THE DRAWINGS

FIG. 1 is a side elevational view of an adult human vertebral column.

FIG. 2 is a posterior elevational view of the column of FIG. 1 and depicting a system according to an embodiment of the invention.

FIG. 3 is an elevational view of one of the vertebra of the column of FIGS. 1 and 2.

FIG. 4 is an enlarged view of a portion of the column of FIGS. 1 and 2 and the system of FIG. 2.

FIG. 5 is an enlarged isometric view of a dampening mechanism of the system of FIGS. 2 and 4.

FIG. 6 is a cross-sectional view of the mechanism of FIG. 5.

FIGS. 6A and 6B are views similar to FIG. 6, on a reduced scale, depicting the movements of the dampening mechanism.

FIG. 7 is an exploded view of an alternate embodiment of the mechanism of FIG. 6.

FIG. 8 is a cross-sectional view of the mechanism of FIG. 7.

FIGS. 8A and 8B are views similar to FIG. 8, on a reduced scale, depicting the movements of the dampening mechanism.

### DETAILED DESCRIPTION

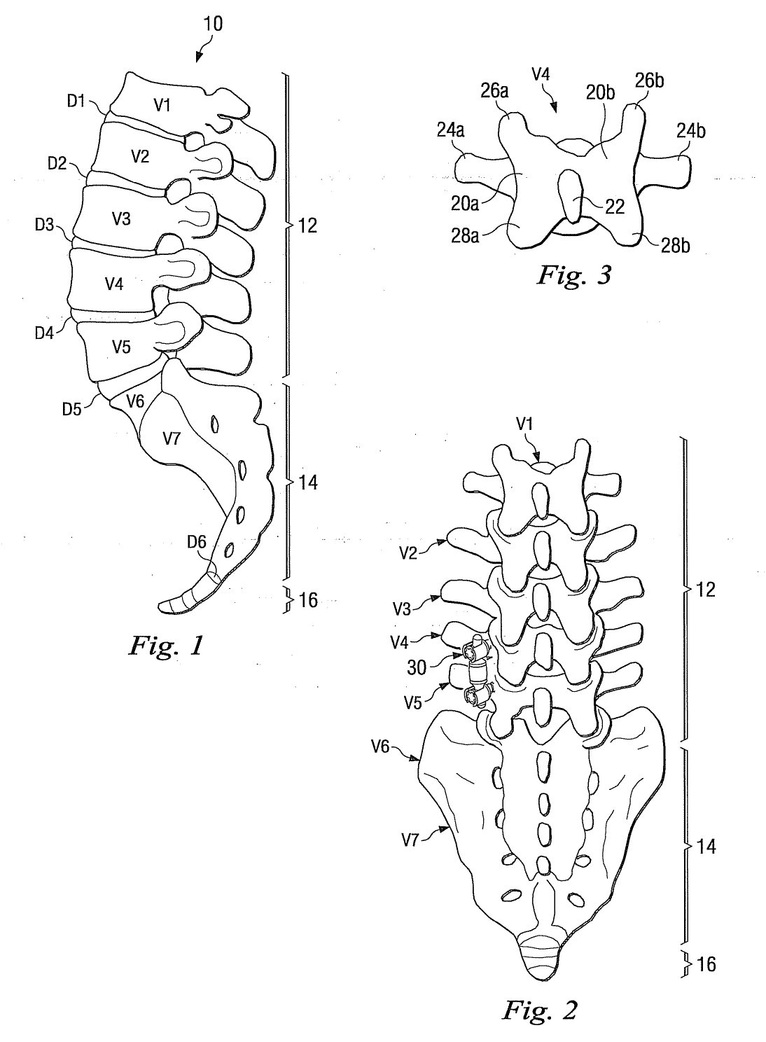

With reference to FIGS. 1 and 2, the reference numeral 10 refers, in general, to the lower portion of a human vertebral column. The column 10 includes a lumbar region 12, a sacrum 14, and a coccyx 16. The flexible, soft portion of the column 10, which includes the thoracic region and the cervical region, is not shown.

The lumbar region 12 of the vertebral column 10 includes five vertebrae V1, V2, V3, V4 and V5 separated by intervertebral discs D1, D2, D3, and D4, with the disc D1 extending between the vertebrae V1 and V2, the disc D2 extending between the vertebrae V2 and V3, and the disc D3 extending between the vertebrae V3 and V4, and the disc D4 extending between the vertebrae V4 and V5.

The sacrum 14 includes five fused vertebrae, one of which is a superior vertebra V6 separated from the vertebra V5 by a disc D5. The other four fused vertebrae of the sacrum 14 are referred to collectively as V7. A disc D6 separates the sacrum 14 from the coccyx 16, which includes four fused vertebrae (not referenced).

With reference to FIG. 3, the vertebra V4 includes two laminae 20a and 20b extending to either side (as viewed in FIG. 2) of a spinous process 22 that extends posteriorly from the juncture of the two laminae. Two transverse processes 24a and 24b extend laterally from the laminae 20a and 20b, respectively; and two articular processes 28a and 28b extend inferiorly from the laminae 20a and 20b, respectively. The inferior articular processes 28a and 28b rest in the superior articular process of the vertebra V5 (FIG. 5) to form a facet joint. Since the vertebra V1-V3 and V5 are similar to the vertebra V4, and since the vertebrae V6 and V7 are noninvolved in the present invention, they will not be described in detail.

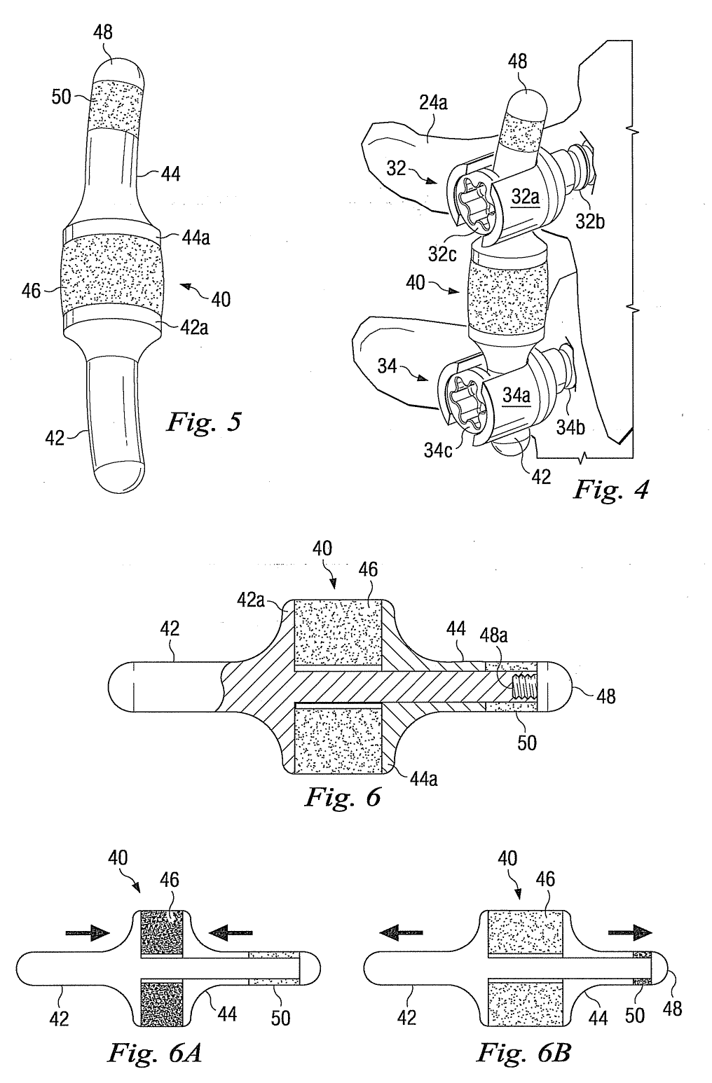

It will be assumed that, for one or more of the reasons set forth above, the vertebra V4 and/or V5 are not being adequately supported by the disc D4 for one or more of the above reasons, and that it is therefore necessary to provide supplemental support and stabilization of these vertebrae. To this end, a system 30 is provided that is shown in FIG. 2 and in greater detail in FIG. 4.

Referring to FIG. 4, the system 30 includes a fixation device, in the form of a screw 32, that is fastened to the vertebra V4; and a fixation device, in the form of a screw 34, that is fastened to the vertebra V5. It is understood that the screws 32 and 34 can be fastened to various areas of the vertebrae V4 and V5 including, but not limited to, the processes, the laminae, or the pedicles.

The screw 32 has a head 32a extending from an externally threaded shank 32b that is screwed in the vertebra V4, and the screw 34 has a head 34a extending from an externally threaded shank 34b that is screwed in the vertebra V5. Each head has a bore, or through opening, extending therethrough, and two set screws 32c and 34c are provided in the heads 32b and 34b, respectively, that can be torqued to secure a member in each opening, as will be described.

Referring to FIGS. 4 and 5, a dampening mechanism 40 is provided that is mounted to the screws 32 and 34. The mechanism 40 has a slight overall curvature and includes a rod 42, and end portion of which extends in the above opening in the screw 32. The set screw 32c is torqued over the rod 42 as necessary to secure the rod 42 to the screw 32.

A tubular member 44 is also provided, and as shown in FIG. 6, a portion of the rod 42 extends through the bore of the tubular member 44, with the corresponding end portion of the rod projecting from the tubular member. An annular flange 42a projects radially outwardly from the rod 42 between its respective ends, and an annular flange 44a projects radially outwardly from one end of the tubular member 44. The flange 44a projects radially outwardly from one end of the tubular member 44. The flange 44a extends in a spaced relation to the flange 42a.

A ring-shaped dampening member 46 extends around the rod 42 and between the flanges 42a and 44a and approximately mid-way between the screws 32 and 34. The dampening member 46 is fabricated from a material having appreciable and conjoint viscous and elastic properties. The axial length of the damping member 46 is greater than that of the damping member 50 so as to have different dampening properties.

A cap 48 has an externally threaded shank 48a that is threadedly engaged with a corresponding internally threaded bore in the other end portion of the rod 42. The diameter of the cap 48 is greater than that of the rod 42 so as to define, with the corresponding end of the rod, an annular space. A ring-shaped dampening member 50 extends around the rod 42 and in the latter space. The dampening member 50 is fabricated from a material having appreciable and conjoint viscous and elastic properties.

A portion of the member 44 extends in the opening in the screw 32, and the length of the member 44 is greater than the diameter of the screw 32 so that the cap 48 and the dampening member 50 extend outside of the opening in the screw. The set screw 34c is torqued over the latter portion of the member 44 as necessary to secure the tubular member 44 to the screw 32.

The mechanism 40 is shown in FIG. 6 in its unloaded state, i.e., when there is no appreciably tensile or compression loads on the vertebrae V4 and/or V5. However when there is flexion or extension of the column 10 caused by corresponding movements of the patient, the mechanism 40 will respond to the resulting compressive and tensile loads on the vertebrae V4 and V5 as follows.

Compressive loads on the vertebrae V4 and V5 causes relative movement of the screws 32 and 36 (FIG. 4) towards each other. This causes relative movement of the rod 42 and the member 44, and therefore the flanges 42a and 44a, towards each other and compresses the dampening member 46, as shown in FIG. 6A, to dampen the movement. After the compressive load and the above relative movements of the screws 32 and 34 towards each other cease, the dampening member 46 will tend to return to its original, non-compressed state, causing relative movement of the flanges 42a and 44a, and therefore the rod 42 and the member 44, away from each other so that the system 30 returns to the unloaded position of FIG. 6.

Relative movement of the screws 32 and 34 away from each other in response to tensile loads on the vertebrae V4 and V5 causes relative movement of the rod 42 and the tubular member 44 away from each other. This causes relative movement of the cap 48 and the member 44 towards each other and thus compresses the dampening member 50 to dampen the movements, as shown in FIG. 6B. After the tensile load and the above relative movements of the screws 32 and 34 away from each other cease, the dampening member 50 will tend to return to its original, non-compressed state and move the cap 48 and the member 44 away from each other so that the system 30 takes the unloaded position of FIG. 6.

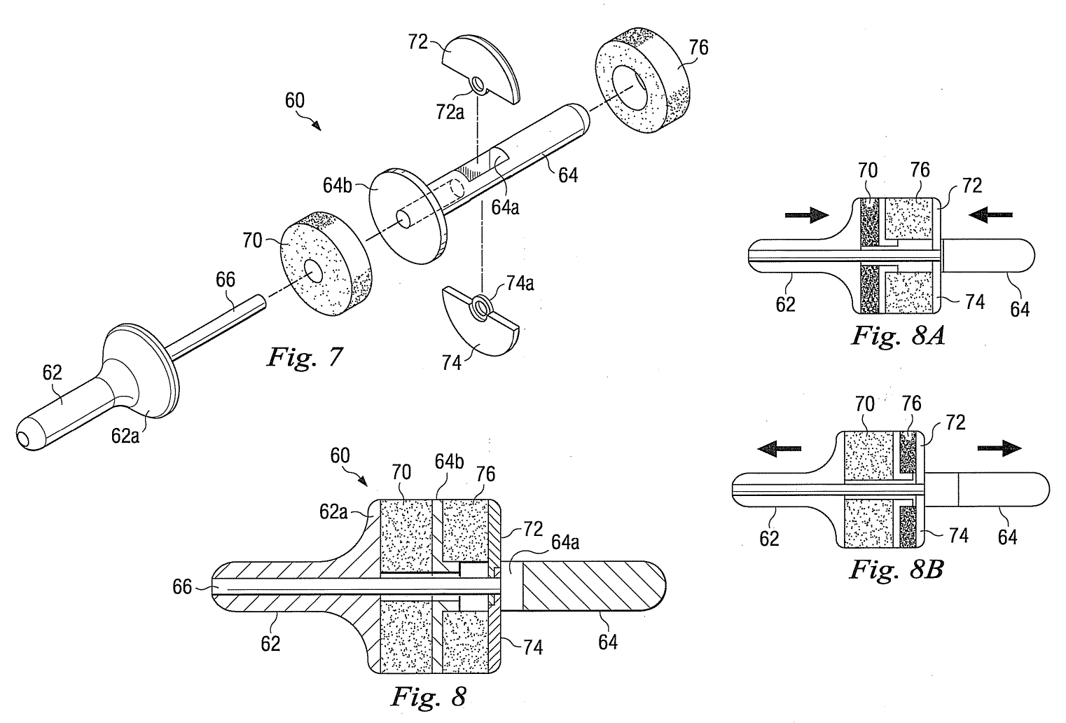

According to the embodiment of FIGS. 7 and 8, a system is provided that includes the screws 32 and 36 (FIG. 4) of the previous embodiment along with a dampening mechanism 60 that is mounted to the screws. In particular, the mechanism 60 includes two axially aligned and spaced rods 62 and 64, with an end portion of the rod 62 extending in the screw 32 and an end portion of the rod extending in the screw 34. The set screws 32c and 34c can be torqued as necessary to secure the rod 62 and the tubular member 64 to the screws 32 and 34, respectively.

A stem 66 extends through a bore formed through the rod 62 and is secured in the bore in any conventional manner. One end of the stem 66 extends flush with the corresponding end of the rod 62, and a portion of the stem 66 projects from the latter rod. A bore is formed in the corresponding end of the rod 64 into which the other end portion of the stem extends.

An annular flange 62a projects radially outwardly from the other end of the rod 62, and an annular flange 64b projects radially outwardly from the other end of the rod 64 and extends in a spaced relation to the flange 62a. A ring-shaped dampening member 70 extends around the stem 66 and between the flanges 62a and 64b. The dampening member 70 is fabricated from a material having appreciable and conjoint viscous and elastic properties.

Two substantially semi-circular plates 72 and 74 are provided with interlocking ring portions 72a and 74a, that are interlocked in the notch 64a and are connected to the corresponding end portion of the stem 66 in any conventional manner. A ring-shaped dampening member 76 extends around the corresponding portion of the rod 64 and in the space between the flange 64b and the interlocked plates 72 and 74. The dampening member 76 is fabricated from a material having appreciable and conjoint viscous and elastic properties.

The mechanism 60 is shown in FIG. 8 in its unloaded state, i.e., when there is no appreciable tensile or compression loads on the vertebrae V4 and/or V5. However, when there is flexion or extension of the column 10 caused by corresponding movements of the patient, the mechanism 60 will respond to the resulting compressive and tensile loads on the vertebrae V4 and V5 as follows.

Compressive loads on the vertebrae V4 and V5 causes relative movement of the screws 32 and 36 (FIG. 4) towards each other. This causes relative movement of the rods 62 and 64, and therefore the flanges 62a and 64b, towards each other and compresses the dampening member 70, as shown in FIG. 8A, to dampen the movement. After the compressive load and the above relative movement of the screws 32 and 36 towards each other cease, the dampening member 70 will tend to return to its original, non-compressed state and cause relative movement of the flanges 62a and 64b, and therefore the rods 62 and 64, away from each other so that the system 30 returns to the unloaded position of FIG. 8.

Relative movement of the screws 32 and 36 away from each other in response to tensile loads on the vertebrae V4 and V5 causes relative movement of the rods 62 and 64, away from each other. This causes movement of the stem 66, and therefore the interlocked plates 72 and 74, relative to the flange 64b in a direction towards each other, thus compressing the dampening member 76 to dampen the movements, as shown in FIG. 8B. After the tensile load and the above relative movement of the screws 32 and 36 away from each other cease, the dampening member 76 will tend to return to its original, non-compressed state and cause relative movement of the stem 66 and therefore the interlocked plates 72 and 74 away from the flange 64b, so the system 30 takes the unloaded position of FIG. 8.

In both of the above embodiments it is understood that as the dampening members 46, 50, 70 and 76 compress in response to the loads on the vertebrae V4 and V5 discussed above, the resistance of the dampening members to the loads will increase with increases in the loads.

### Variations

It is understood that variations may be made in the foregoing without departing for the invention and examples of some variations are as follows:

(1) The systems in each of the above embodiments can be connected to anatomical structures other than vertebrae.

(2) Fixating devices other than the screws described above can be used to connect the dampening mechanisms to the anatomical structures.

(3) The dampening mechanisms in each of the previous embodiments can be rigidly connected at different locations of the vertebrae.

(4) Extra fixation devices, or screws, can be attached to two adjacent vertebrae as shown in the above examples, or to a third vertebrae adjacent to one of the two vertebrae. In each case the rods and/or tubular members described above would be long enough to extend to the extra screws.

(5) In the event that one or more extra fixation devices, or screws, are attached to the vertebrae, an extra dampening mechanism can be attached between the extra fixation device and its adjacent screw.

(6) The dampening members disclosed above can be fabricated from materials other than those described above and many include a combination of soft and rigid materials other than those described above and may include a combination of soft and rigid materials.

(7) The dampening properties of the dampening member 46 and 50 can be varied in manners other than providing them with different axial lengths, such as fabricating them from different materials, etc.

(8) One or more of the components disclosed above may have through-holes formed therein ti improve integration of the bone growth.

(9) The components of one or more of the above embodiments may vary in shape, size, composition, and physical properties.

(10) Through-openings can be provided through one or more components of each of the above embodiments to receive tethers for attaching the devices to a vertebra.

(11) The systems of each of the above embodiments can be placed between two vertebrae in the vertebral column other than the ones described above.

(12) The systems of the above embodiments can be inserted between two vertebrae following a discectemy in which a disc between the a adjacent vertebrae is removed, or corpectomy in which at least one vertebrae is removed.

(13) The spatial references made above, such as “under”, “over”, “between”, “flexible, soft”, “lower”, “top”, “bottom”, “axial”, “transverse”, etc., are for the purpose of illustration only and do not limit the specific orientation or location of the surface described above.

The preceding specific embodiments are illustrative of the practice of the invention. It is to be understood, therefore, that other expedients known to those skilled in the art or disclosed herein, may be employed without departing from the invention or the scope of the appended claims, as detailed above. In the claims, means-plus-function clauses are intended to cover the structures described herein as performing the recited function and not only structural equivalents but also equivalent structures. Thus, although a nail and a screw may not be structural equivalents in that a nail employs a cylindrical surface to secure wooden parts together, whereas a screw employs a helical surface, in the environment of fastening wooden parts a nail and a screw are equivalent structures.

What is claimed is:

1. A system for connecting two anatomical structures, the system comprising:
a first fixation device affixed to one of the structures;
a second fixation device affixed to the other structure; and
a dampening mechanism comprising:
a first member connected to the first fixation device and having a first flange;
a second member connected to the second fixation member and having a second flange extending in a spaced relation to the first flange;
a first dampening member extending between the flanges so as to dampen any relative movement of the first and second members towards each other;
a third member connected to the first member and defining with the second member a space; and
a second dampening member extending in the space so as to dampen any relative movement of the first and second members away from each other.

2. The system of claim 1 wherein the second dampening member dampens movement between the third and second members, and therefore between the first and second members.

3. The system of claim 1 wherein the first dampening member and the second dampening member having different dampening properties.

4. The system of claim 3 wherein the first dampening member and the second dampening have different dimensions and therefore different dampening properties.

5. The system of claim 1 wherein relative movement between the first member and the second member towards each other causes compression of the first dampening member, and wherein relative movement of the first member and the second member away from each other causes compression of the second dampening member.

6. The system of claim 5 wherein the relative movement of the first member and the second member away from each other causes relative movement of the third member and the second member towards each other.

7. The system of claim 1 wherein the first dampening member extends mid-way between the fixation devices.

8. The system of claim 1 wherein the first member is a rod and rod wherein the first flange extends radially outwardly from the rod.

9. The system of claim 8 wherein the second member is a tubular member and wherein the second flange extends radially outwardly from the tubular member.

10. The system of claim 9 wherein a portion of the rod extends in the bore of the tubular member and wherein the first dampening member is in the form of a ring that extends around the rod and between the flanges.

11. The system of claim 10 wherein the third member is a cap that is connected to the rod and extends in a spaced relation to the tubular member to define the space.

12. The system of claim 11 wherein one end portion of the rod is connected to a fixation device and wherein the cap is connected to the other end of the rod.

13. The system of claim 12 wherein the length of the tubular member is greater than the corresponding dimension of the other fixation device so that the cap and the second dampening member extend outside of the other fixation device.

14. The system of claim 5 wherein the second member is a rod, and wherein the second flange extends radially outwardly from the latter rod.

15. The system of claim 14 wherein a bore is formed in each of the rods and further comprising a stem extending through the bores.

16. The system of claim 15 wherein the stem is affixed to the first-mentioned rod and moves relative to the second-mentioned rod.

17. The system of claim 14 wherein the first dampening member is ring-shaped and extends around the stem.

18. The system of claim 14 wherein the third member comprises two interlocked, substantially semicircular plates extending radially outwardly from the stem.

19. The system of claim 18 further comprising a notch formed in the second-mentioned rod and wherein the plates are interlocked in the notch.

20. The system of claim 18 wherein the space is defined between the second flange and the interlocked plates.

21. The system of claim 18 wherein the second dampening member extends around the second-mentioned rod.
